(19) United States
(12) Patent Application Publication
(10) Pub. No.: US 2026/0234538 A1
(43) Pub. Date: Aug. 13, 2026

(54) PROCESS AND EQUIPMENT FOR EFFECTING LYSIS OF CELLS

(71) Applicant: WHOLE GREEN FOODS PTY LTD, Mount Claremont, Western Australia (AU)

(72) Inventor: Nicholas Emmanuel STAMATIOU, Hillarys, Western Australia (AU)

(21) Appl. No.: 19/155,251

(22) PCT Filed: Feb. 9, 2024
(86) PCT No.: PCT/AU2024/050086

§ 371 Date: Aug. 10, 2025

(30) Foreign Application Priority Data
Feb. 9, 2023 (AU) 2023900310

(51) Int. Cl. C12M 1/00; A23C 11/10; A23L 7/10; C12M 1/26; C12M 1/33

(52) U.S. Cl. CPC C12M 47/06; A23C 11/10; A23L 7/10; C12M 33/12; C12M 45/02

(57) ABSTRACT

A process for effecting lysis of cells of plants material or parts thereof; the process incorporating the steps of: obtaining the plant material at a target particle size; combining the plant material in a fluid medium; subjecting the plant material to a set of predetermined processing conditions; deaerating the plant material thereby to substantially remove gases; introducing the plant material into a two-stage compression device, within which it is subjected to a first pressure in a high fluid pressure compression first stage, and subsequently to a second pressure in a rapid decompression second stage, wherein the first pressure is substantially higher than the second pressure thereby subjecting the plant material to cavitation of water inside the cell wall causing cell wall rupture.

### TECHNICAL FIELD

The invention relates to the field of plant cell lysis technology. In particular, the invention relates to an improved process and equipment for effecting lysis of plant cells to extract functional and nutritional compounds from plant-derived materials.

### BACKGROUND OF THE INVENTION

Plants materials, including for example grains, legumes, vegetables, grasses, fibres, and nuts, have long been recognized as rich sources of functional and nutritional compounds. These compounds, which include proteins, carbohydrates, lipids, vitamins, and minerals, contribute to the nutritional value of the plant materials and may also have beneficial effects on human health. However, these compounds are often located inside the cells of the plant materials are therefore not readily accessible.

Various processes have been developed to break down the cell walls of plant materials and release the functional and nutritional compounds. These processes typically involve physical disruption of the cells, such as cutting, grinding, soaking, heating, or mashing. While these processes can effectively break down the cell walls and release the compounds, they can also cause damage to the compounds and reduce their functionality and nutritive value. For example, heating can denature proteins and destroy vitamins, while excessive shear can cause physical damage to the compounds.

Another challenge in processing plant materials is the presence of entrained gases in the plant material slurry. These gases can cause problems in downstream processing steps, such as frothing of the slurry or damage to processing equipment. Therefore, it is often desirable to remove or reduce the entrained gases prior to further processing. This is typically achieved by deaeration, which involves subjecting the slurry to a reduced pressure to allow the gases to vent.

High pressure homogenization is a technique that has been used to disrupt cells and release the functional and nutritional compounds (as well as to create more stable emulsions). In this process, the plant material slurry is subjected to a high pressure, which causes the cells to burst and release the compounds. However, conventional high pressure homogenization processes typically involve a single stage of compression, and the pressure is not sufficiently high to effectively (and homogeneously) burst the cells without causing damage to the compounds and/or the equipment.

Therefore, there is a continuing interest in developing improved processes for extracting functional and nutritional compounds from plant materials. These processes ideally would allow for effective cell lysis without causing substantial damage to the compounds and/or equipment, and would also effectively remove or reduce entrained gases in the plant material slurry.

### SUMMARY OF THE INVENTION

According to a first aspect of the invention, there is provided a process for effecting lysis of cells of plants material or parts thereof; the process comprising: obtaining the plant material at a target particle size; combining the plant material in a fluid medium, optionally incorporating other desired chemicals or agents, in a vessel; subjecting the plant material to a set of predetermined processing conditions; deaerating the plant material thereby to substantially reduce or remove entrained gases; and introducing the plant material into a two-stage compression device, within which it is subjected to a first pressure in a high fluid pressure compression first stage, and subsequently to a second pressure in a rapid decompression second stage, wherein the first pressure is substantially higher than the second pressure thereby subjecting the plant material to cavitation of water inside the cell wall causing cell wall rupture.

The target particle size of the plant material may range between 400 and 2000 microns or, more preferably, between 400 and 800 microns. Known techniques for preliminary reduction of particle size of the plant material may be used such as, for example, milling, grinding, chopping, pulverization etc.

The set of predetermined processing conditions may be dependent on (or optimised to) the raw materials used and include one or more of: temperature, temperature profile, pH, viscosity, and time. The set of predetermined processing conditions may include a temperature below 60C for certain plant materials that are susceptible to degradation of flavour and/or nutrition. However, it should be appreciate that this is not a physical or technical limitation of the process.

The deaeration action and compression process may occur concurrently via a partial recycling of compressed material into the vessel from the outlet of the deaeration device. In a representative embodiment, the compression device comprises one or more pistons in a cylinder, through which the fluid passes. In a further representative embodiment, the compression device comprises three, five, seven, or nine, pistons in a cylinder, through which the fluid passes.

The inventors have also found that the deaeration step carried out prior to the compression step effectively removes entrained gases from the slurry of plant material. This is particularly advantageous as this allows the achievement of higher pressures in the two-stage compression device and more consistent cell bursting (i.e., lysis or cavitation of the cells) in the compression stage, as well as preventing other issues such as auto-oxidation of any fatty acids present.

The inventors have found that the pressure shock to the cell walls provided by the sudden drop in pressure (i.e., rapid decompression) from the first to the second stage of the two-stage compression device causes the cells to burst (i.e., lyse) effectively, without causing denaturation or other damage to the compounds within the cells, thus rendering them more functional and bioavailable in comparison with other processes for the destruction or disruption of plant cells.

Without wishing to be bound by a specific theory of operation, the inventors postulate that the higher fluid pressure initial stage of compression causes an increase in fluid pressure outside the cell, thereby tending to force surrounding fluid into the cell. The sudden and precipitous drop in pressure (resulting from the rapid decompression) outside of the (thus engorged) cell causes the fluid inside the cell to expand, due to the now-large pressure gradient across the cell wall, tending to burst the cell wall open to equalise the fluid pressure.

The first pressure may be between 400 Bar and 3000 Bar and more preferably between 900 Bar and 2000 Bar. At 900 Bar and above, sterilisation of the plant material may be effected at relatively lower temperatures (e.g. lower than 60° C.) compared with other forms of cell lysis, further enhancing the preservation of the nutritive and functional compounds released from the cells.

The second pressure may be between atmospheric pressure and 400 Bar.

In order to provide a sufficient pressure drop to effect cell lysis via cavitation, the ratio of the first pressure to the second pressure may be 4:1 or greater. For example, the first pressure is approximately 900 Bar and the second pressure is approximately 200 Bar. However, it is preferable that the first pressure be at least 400 Bar and above in order to achieve efficient cell lysis.

In other useful embodiments, the first pressure is in the range of approximately 600-1500 Bar and the second pressure is in the range of approximately 0-350 Bar.

The deaeration device may comprise a substantially vertical pressure vessel having an outer tube and an open inner annular tube, wherein the inner tube extends between 65% and 75% of the length of the outer tube from an inlet end of the deaeration device; and wherein the plant material entering the deaeration device is directed internally of the inner tube.

According to another aspect of the invention, there is provided a two-stage high fluid pressure compression device for effecting lysis of cells of plant material, the device comprising: a first stage for subjecting the plant material to a first pressure; and a second stage for subjecting the plant material to a second pressure that is substantially lower than the first pressure, wherein the pressure differential between the first and second stages causes cavitation of water inside the cell wall of the plant material, leading to cell wall rupture.

The first pressure may be between 400 Bar and 3000 Bar and more preferably between 900 Bar and 2000 Bar. At 900 Bar and above, sterilisation of the plant material may be effected at relatively lower temperatures (e.g. lower than 60° C.) compared with other forms of cell lysis, further enhancing the preservation of the nutritive and functional compounds released from the cells.

The second pressure may be between atmospheric pressure and 400 Bar.

In order to provide a sufficient pressure drop to effect cell lysis via cavitation, the ratio of the first pressure to the second pressure may be 4:1 or greater. For example, the first pressure is approximately 900 Bar and the second pressure is approximately 200 Bar. However, it is preferable that the first pressure be at least 400 Bar and above in order to achieve efficient cell lysis.

In other useful embodiments, the first pressure is in the range of approximately 600-1500 Bar and the second pressure is in the range of approximately 0-350 Bar.

The two-stage high fluid pressure compression device may be coupled at its inlet to a deaeration device that comprises a substantially vertical pressure vessel having an outer tube and an open inner annular tube, wherein the inner tube extends between 65% and 75% of the length of the outer tube from an inlet end of the deaeration device; and wherein the plant material entering the deaeration device is directed internally of the inner tube.

According to another aspect of the invention, there is provided the use of a two-stage high fluid pressure compression operation to cause lysis of cell walls of plant material entrained in a fluid medium, via cavitation of water inside the cells, wherein the first stage of the two stages is conducted at a (first) pressure that is substantially higher than the (second) pressure of the second stage, thereby subjecting the plant material to cavitation of water inside the cell wall causing cell wall rupture.

Preferably, the compression is effected via a device comprising one or more pistons in a cylinder, through which the fluid passes. Preferably, the compression operation is conducted subsequent to, or concurrently with, deaeration of the plant material.

According to another aspect of the invention, there is provided a two stage high pressure compression device adapted to cause lysis of cell walls of plant material entrained in a deaerated fluid slurry, when the plant material passes therethrough, wherein the first stage of the two stages is conducted at a (first) pressure that is substantially higher than the (second) pressure of the second stage thereby subjecting the plant material to cavitation of water inside the cell wall causing cell wall rupture.

Preferably, the compression device is coupled at its inlet to a deaeration device that comprises a pressure vessel having an outer tube and an open inner annular tube, wherein the inner tube extends between 65% and 75% of the length of the outer tube from an inlet end of the device; and wherein the plant material entering the device is directed internally of the inner tube.

According to another aspect of the invention, there is provided nutritional compounds obtained from plant materials via the process described above.

According to another aspect of the invention, there is provided a food product comprising plant material processed by a method comprising: obtaining the plant material at a target particle size; combining the plant material in a fluid medium in a vessel; subjecting the plant material to a set of predetermined processing conditions; deaerating the plant material (and/or plant material slurry) to substantially reduce or remove entrained gasses; and introducing the plant material into a two-stage compression device, within which it is subjected to a first pressure in a high fluid pressure compression first stage, and subsequently to a second pressure in a rapid decompression second stage, wherein the first pressure is substantially higher than the second pressure thereby subjecting the plant material to cavitation of water inside the cell wall causing cell wall rupture.

The target particle size of the plant material may range between 400 and 2000 microns or, more preferably, between 400 and 800 microns. Known techniques for preliminary reduction of particle size of the plant material may be used such as, for example, milling, grinding, chopping, pulverization etc.

The first pressure may be between 400 Bar and 2000 Bar, and the second pressure may be between atmospheric and 400 Bar.

According to another aspect of the invention, there is provided a food product incorporating the lysed plant material obtained by the process described above.

Now will be described, by way of specific, non-limiting examples, preferred embodiments of the invention with reference to the drawings.

### BRIEF DESCRIPTION OF THE DRAWINGS

FIG. 1 is a flowchart representing a preferred embodiment the process according to the invention

FIG. 2 is a schematic top view of a plant for carrying out the process according to the invention.

FIG. 3 is a photograph of a hammer mill suitable for the size reduction step of the present invention.

FIG. 4 is a schematic top view of a plant for carrying out the process according to the invention.

FIG. 5 is a photograph of a bioreactor suitable for use in the invention.

FIG. 6 is a schematic top view of a plant for carrying out the process according to the invention.

FIG. 7 is a photograph of a typical plate heat exchanger as per the type used on the inventive process.

FIG. 8 is a schematic top view of a plant for carrying out the process according to the invention.

FIG. 9 illustrates a deaeration tube for use in the present invention.

FIG. 10 is a schematic top view of a plant for carrying out the process according to the invention.

FIG. 11 is a section schematic view of a two-stage homogeniser head according to the invention.

### DETAILED DESCRIPTION OF THE INVENTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The process and equipment described herein may offer several advantages. For instance, the process may allow for the extraction of functional and nutritional compounds from plant materials in a manner that does not substantially lower their nutritive value or functionality. This is achieved by avoiding denaturing of the compounds at higher temperatures or damaging their structure with excessive shear, which are common issues associated with traditional plant material processing methods. Furthermore, the process may be particularly beneficial for the production of plant-based milk or concentrates, providing a product that performs sufficiently in comparison to dairy milk with a reduced or eliminated the need for further functional additives.

The present disclosure relates to a process and equipment for effecting lysis of cells of plant material. In particular, the disclosure may provide a method for obtaining functional and nutritional compounds from plant-derived materials, such as for example grains, legumes, fruits, vegetables, grasses, fibres, and nuts. The process may involve obtaining the plant material at a target particle size, combining the plant material in a fluid medium, optionally incorporating other desired chemicals or agents, and subjecting the plant material to a set of predetermined processing conditions. The plant material may then be deaerated to substantially reduce or remove entrained gases, and subsequently introduced into a two-stage compression device, within which it is subjected to a first pressure in a high fluid pressure compression first stage, and subsequently to a second pressure in a rapid decompression second stage, wherein the first pressure is substantially higher than the second pressure. This process (and particularly the rapid and explosive decompression) preferably results in the cavitation of water inside the cell wall, causing cell wall rupture and the release of the desired compounds.

The invention in one embodiment is a process for achieving the bursting or lysis of edible plant cell material, at relatively low temperatures (e.g. below 60° C.), thereby releasing functional and/or nutritional compounds that are relatively non-degraded in comparison with known prior art processes. In an alternative embodiment, the invention is a process and equipment for achieving a high throughput of lysed plant material with a mean particle size of between 1 and 40 micron. For example, the above described process and equipment is capable of producing upward of 1,000 L/hr of lysed plant material (in a slurry solution), and optimally between 2,000 and 10,000 L/hr of lysed plant material depending on the plant material input used.

The key to the process is to submit the plant cells (prepared as a wet slurry) to a two-stage compression process where the first stage operates at a relatively higher pressure, followed immediately by a second lower compression stage that operates at a relatively lower pressure. The immediate pressure drop (i.e., rapid or explosive decompression) causes the cell walls to burst and release various compounds from within the cell that then become relatively more functional and/or more bioavailable for use as or in food ingredients compared with processes that cause greater levels of damage to these compounds, often through generation of higher temperatures that tend to denature the compounds.

Typically, the first pressure is between 400 Bar and 3000 Bar and more preferably between 900 Bar and 2000 Bar. At 900 Bar and above, sterilisation of the plant material can be effected at a relatively lower temperatures (e.g. lower than 60° C.) compared with other forms of cell lysis, further enhancing the preservation of the nutritive and functional compounds released from the cells. Typically, said the second pressure is between atmospheric pressure and 400 Bar.

Typically, in order to provide a sufficient pressure drop to effect cell lysis via cavitation, the ratio of the first pressure to the second pressure is 4:1 or greater. For example, and by no means limiting the scope of the invention, the first pressure may be approximately 900 Bar and the second pressure may be approximately 200 Bar. However, the specific pressures will depend on one or more of: the desired particle size of the output plant material slurry, the desired throughput of the equipment and process; the level of sterilisation required in the output plant material slurry, the required degree of total lysis of cells in the plant material, and the degree of homogenisation of the output plant material slurry sought.

In other useful embodiments, the first pressure is in the range of approximately 600-1500 Bar and the second pressure is in the range of approximately 0-350 Bar.

It is particularly advantageous where the process provides a step prior to the two-stage compression device wherein the plant material slurry is substantially deaerated to prevent excess foaming or other deleterious processes to occur in the output plant material slurry. Additionally, and in light of the significant pressures used in the two-stage compression device, the de-aeration is highly desirable to prevent damage to hydraulics used in the creation of this significant pressure (particularly over a period of extended use). By removing any entrained gasses from the plant material slurry, the two-stage compression device is less likely to encounter air pockets in the plant material slurry that can often cause damage to the equipment (in the case of hydraulic systems with piston assemblies).

The invention also encompasses the above-described two-stage compression operation per se, featuring the above described substantial pressure drop and a compression device adapted to perform this operation.

For a typical plant material to be processed via the inventive process, the major steps involved will typically be as follows (although it should be appreciated that one or more of these steps are not required depending on the specific plant material being processed):

Size Reduction>Aqueous Soaking>Temperature Adjustment>Deaeration>High Pressure Compression>Post processing/Packing.

As indicated above, certain applications (particularly those where ‘soft’ plant materials are processed) will not require an aqueous soaking step, as this is typically more suited to grains and legumes where a liquid is used to soften the plant material prior to milling or size reduction. Similarly, many plant materials will not require temperature adjustment, with elevated temperature typically only being used where enzymes or other processing aids (requiring elevated temperatures) are used.

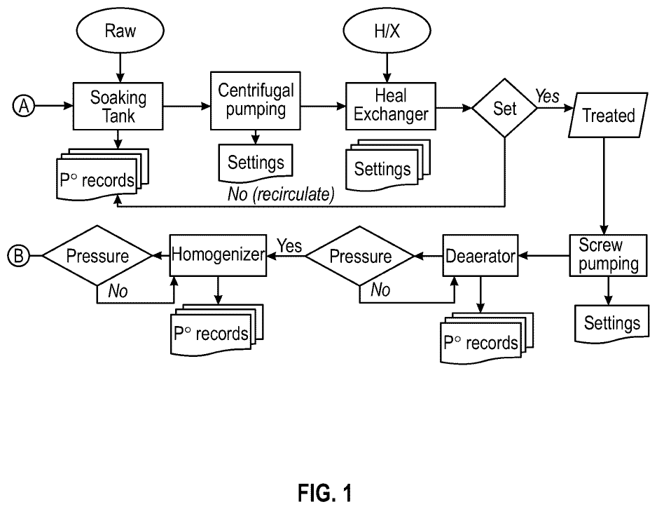

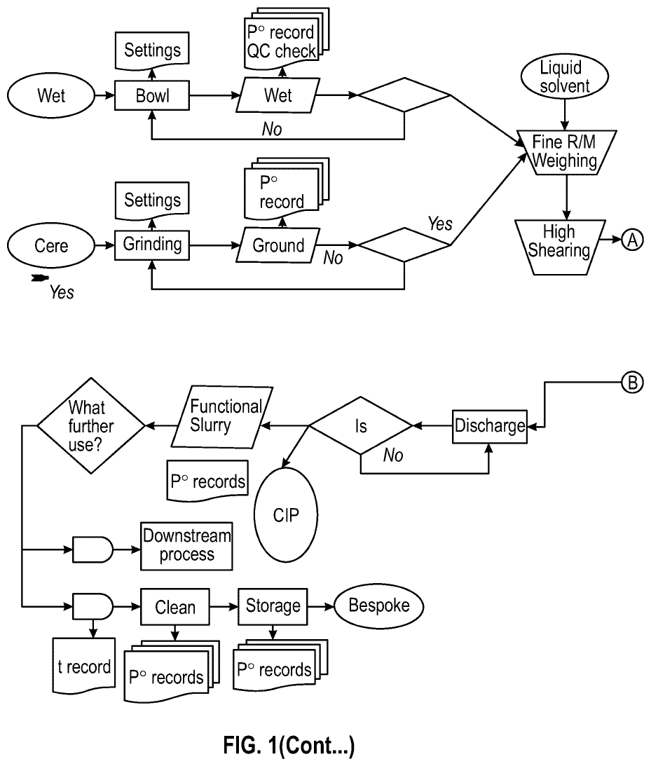

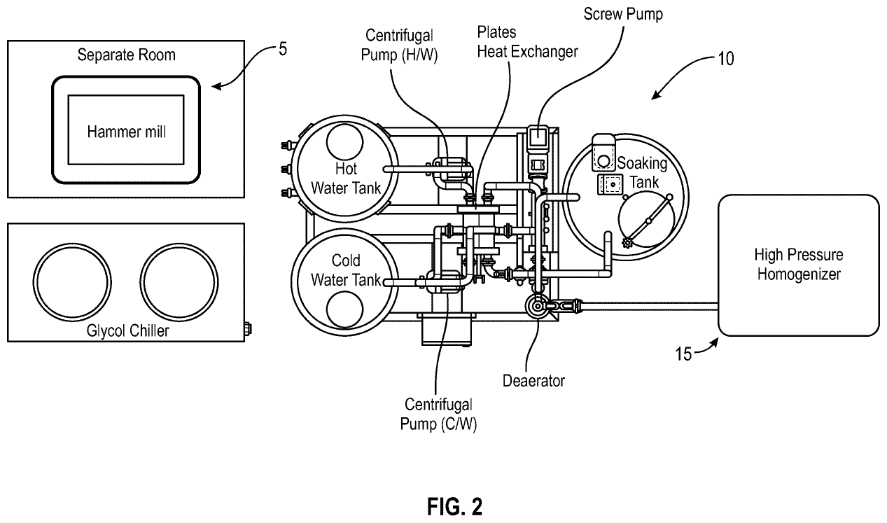

Turning to FIG. 1 there is shown a typical overall process flow for a process according to the invention. FIGS. 2 to 11 show a sample layout for a plan capable of performing the process according to the invention and these will be referred to below in an example describing the inventive process in greater detail.

### Raw Material Size Reduction

Raw material size reduction is the first processing step of the inventive process. The inventive process described works optimally with a feedstock of a slurry of finely ground solid plant material having a particle size of 1.0 mm (1000 microns) or less, preferably less than 0.8 mm (800 microns). However, it should be appreciated that for certain ‘softer’ ingredients, it may be possible to use a feedstock of a slurry with plant material having a particle size of up to 2000 or even 3000 microns.

Depending on the nature of the plant material to be processed, appropriate pre-processing equipment (either wet or dry processing equipment) can be selected to achieve the desired particle size, such as grinders, colloid mills, pulverisers, or hammer mills for dry cereals or bowl choppers for wet products to create a purée. Alternatively, raw materials can be supplied in the appropriate fineness to the processing plant.

If the particle size is too big, the specific surface area (surface area per unit mass) of the material is lower. Consequently, the reaction yield in the downstream bioreactor, and the functionality of the slurry, are therefore also reduced. Large particle sizes are also prone to caking or blocking the downstream plate heat exchanger, causing disruption to production and loss of performance and process control. Also, larger particles may have a deleterious effect on the compression device, as this can lead to rapid wearing of internal parts, or blockage of the aperture in the manifold, which will impact the quality of the output or prevent the plant material slurry from entering the two-stage compression device.

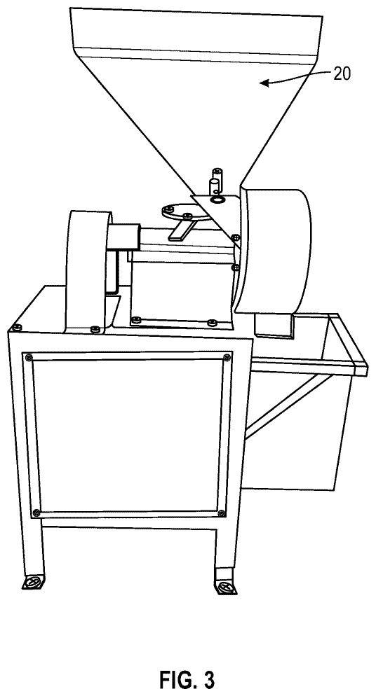

FIG. 2 shows the relative location of process 10 to the two-stage compression device 15 (labelled High Pressure Homogenizer) and hammer mill 5 and grinding operation, and FIG. 3 shows a typical hammer mill 5 with an input chute 20 that may be used.

### Soaking/Steeping of Raw Materials

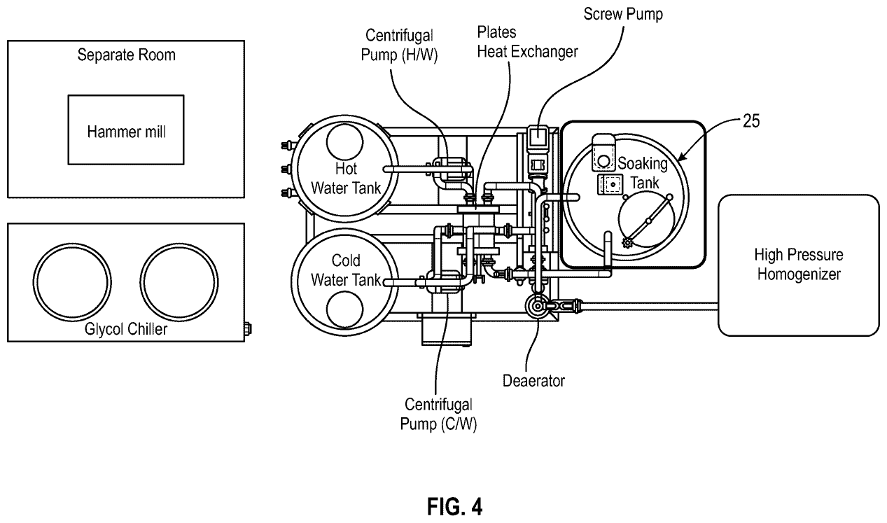

Soaking of the ground raw materials, in this example, is carried out in a 1,000 L tank (bioreactor or soaking tank 25 depending on the specific plant material being processed). The bioreactor or soaking tank 25 is used to soak the ground plant material in water and, if required, to perform other operations to prepare the material for the compression operation, including but not limited to: heating, cooling, enzymatic treatments, pH adjustment. This is typically done to prepare the slurry for further processing and to achieve preliminary functionality. Circled in bold in FIG. 4 is where this operation occurs in the plant in this example.

Depending on the raw materials and the desired output functionality, the soaking may be simply dispersion of the raw materials into cold water, with no specific further operation required (e.g. if processing used coffee grounds, fruit, vegetables, chilli, garlic, fig leaves etc).

The soaking operation in this example is managed in a batch mode, for which different ingredients are precisely weighed out and added as required according to the recipe in use. The bioreactor tank (or soaking tank 25 in the case that no enzymatic processing is required) is mounted with an agitator to get homogenous dispersion of materials and temperature throughout the slurry. Advantageously, a manhole in the top of the bioreactor tank or soaking tank 25 enables the addition of any desired different ingredients at the different stages of the soaking process.

A high shear mixer can be installed in a recirculation mode to ensure the particles size is further standardised. Another particle size standardisation feature can be mounted post-bioreactor, such as a strainer to retain any particle above the critical size.

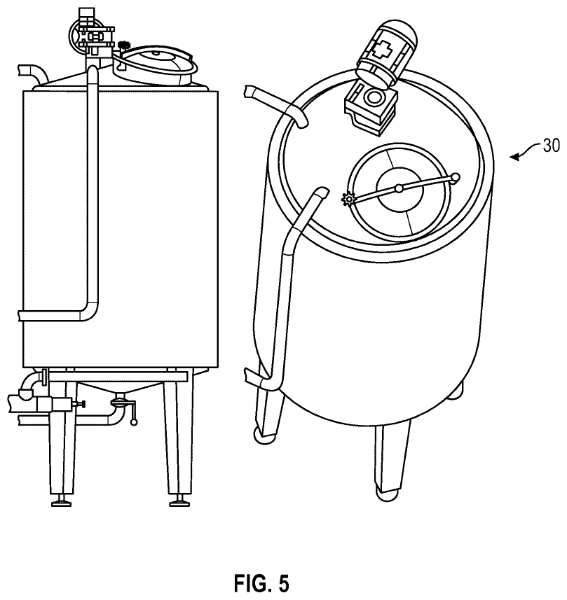

FIG. 5 shows a typical stainless steel bioreactor tank 30 (or soaking tank in the case that no enzymatic processing is required) as per the present example.

### Heat Exchange System

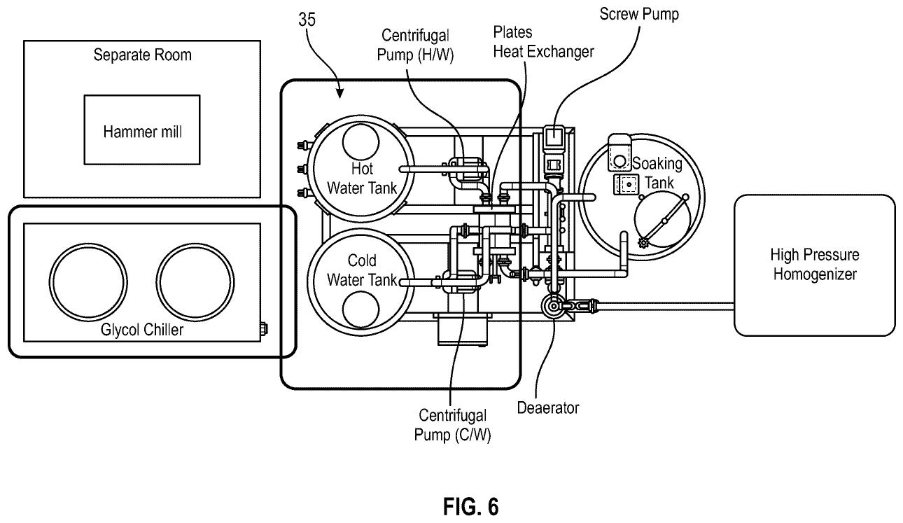

FIG. 6 shows the main components 35 of the heat exchange system and their location relative to the bioreactor (or soaking tank 25). The area circled in bold shows where this operation occurs in the overall process. Typically, the heat exchange process is a service step that can be used in combination with the above soaking step.

There are six components that make up the heat exchange process in this example. The core device is the Plate Heat Exchanger. This is where the plant material slurry and the technical fluid (either hot or cold water) are in thermal contact for the purpose of transferring heat to or from the slurry. A Glycol Chiller is used as a refrigeration unit that cools a buffer tank of cold water/glycol when chilling is required in the heat exchanger. A buffer tank of hot water provides the heating liquid as required by the heat exchange. Each buffer tank is mounted with its own centrifugal pump to circulate its fluid through the heat exchanger.

The cold water circuit can also be used to supply the compression device, as required. This is not a critical requirement, merely an optional optimisation to avoid running lost water to the compression device (default supply).

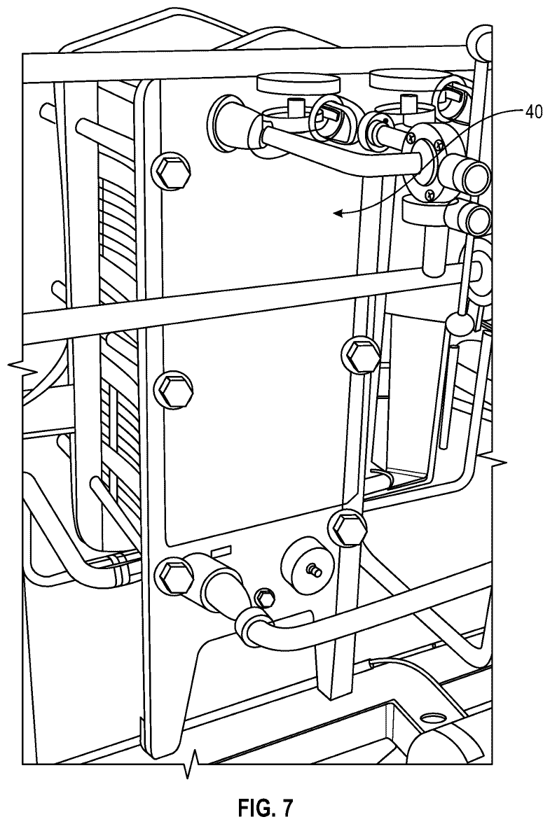

FIG. 7 shows a typical plate heat exchanger 40 of the type used in this example.

### Deaeration

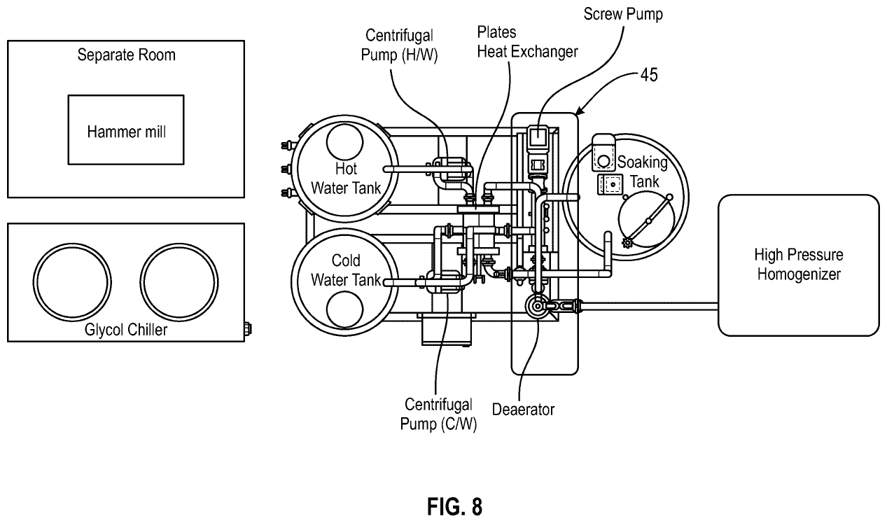

Deaeration is a highly preferred step prior to sending the slurry to the compression device. In this example, it takes place in a bespoke pressure vessel device. Circled in bold in FIG. 8 illustrates where this deaeration operation 45 occurs in the overall process. As described in detail above, in light of the significant pressures used in the two-stage compression device, the de-aeration step is highly desirable to prevent damage to hydraulics used in the creation of this significant pressure (particularly over a period of extended use). By removing any entrained gasses from the plant material slurry, the two-stage compression device is less likely to encounter air pockets in the plant material slurry that can often cause damage to the equipment (in the case of hydraulic systems with piston assemblies).

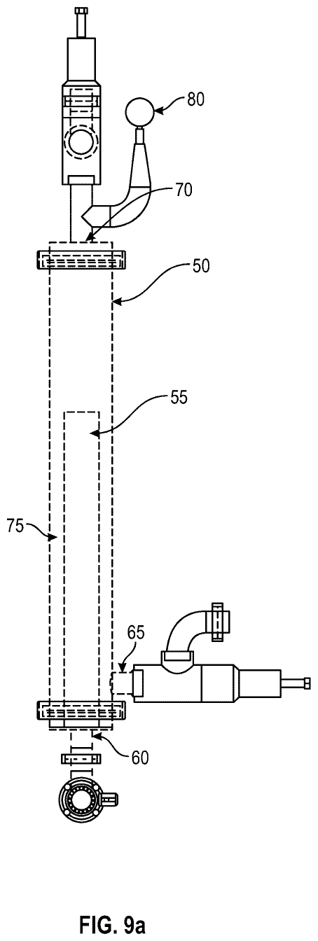

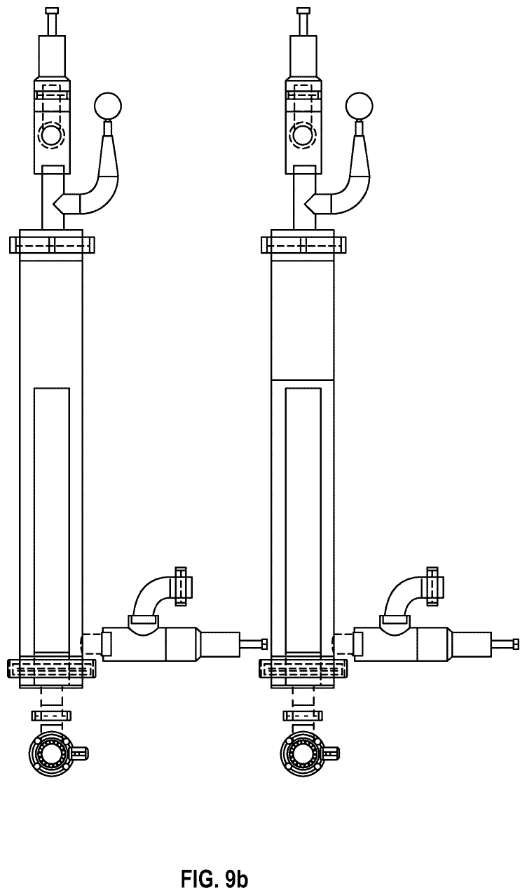

The deaerator is in this example (as shown in FIG. 9a) a tubular pressure vessel having an outer tube 50 and an open inner annular tube 55, wherein said the inner tube 55 extends between 65% and 75% of the length of the outer tube 50 from an inlet 60 end of the device; and wherein the plant material entering the device is directed internally of the inner tube 55, as per FIGS. 9a and 9b. As shown in the figures, the tubular pressure vessel (including outer tube 50 and open inner annular tube 55) are substantially vertical to maximise the operation of gravity such that when the plant material slurry flow through the open inner annual tube 55 via the inlet 60 and overflows into the cavity 75 between the outer tube 50 and inner tube 55, any entrained gasses in the plant material slurry are vented into an upper portion of the outer tube 50 and out of the deaerator device via an outlet 70 (if required)

Once the slurry has been properly prepared and treated in the bioreactor (or soaking tank 25), an eccentric screw pump transfers it through the deaerator device via the open inner annual tube 55 with an internal pressure within the deaerator device of up to 9 Bar. The de-aerator is fitted with two pressure relief gauges, one at the top (recirculation outlet 70), that is linked to the bioreactor or soaking tank 25 recirculation line, and one at the bottom (outlet 65) that is linked to the two-stage compression device inlet.

The operation requires opening the outlet gauge 65 and then slowly closing the recirculation outlet 70 to create the correct equilibrium and back pressure of approximately 2 to 6 Bar against the partially closed inlet valve for a given pump pressure. This will create a constant inverted wedge in the slurry flow, releasing any entrained bubbles, froth and air trapped in the slurry due to the stirring and heat exchanger recirculation in the bioreactor. This is important to achieve higher pressures of over 900 Bar in the compression stage, producing consistent and even cell bursting (i.e., cell lysis) in the two-stage compression device (i.e., high pressure homogeniser).

For example, removing air from the slurry helps to avoid the oxidation of the slurry and prevent ‘staleness’ flavour notes and other non-desirable reactions. Degassing also facilitates downstream packaging by minimising foaming of the compressed product. Also, if the slurry is not deaerated, the compression device can be damaged and worn due to the very high pressures generated in the lysing process. It also guarantees a constant back pressure for the compression device.

FIG. 9a illustrates a deaeration tube as per the present example, FIG. 9b illustrates the operation of the deaeration system.

In FIG. 9a, the deaerator consists of an outer tube 50 that is pressure-sealed at each end. There is an inner tube 55 that extends approximately 65% of the length of the outer tube 50 form the inlet 60 end. However, it should be appreciated that in alternative embodiments, the inner tube 55 may extend between approximately 50% and 90% of the length of the outer tube 50 from the inlet 60. The slurry inlet 60 is located at the bottom of the deaerator and arranged so that the slurry enters through the bottom of the inner tube 55. There are two outlets-one outlet 65 to the two-stage compression device and one outlet 70 to the recirculation circuit back to the bioreactor or soaking tank 25. The outlet 65 to the two-stage compression device is located also at the bottom of the deaerator and taps into the annular space 75 between the inner 55 and outer 50 tubes, while the recirculation outlet 70 is at the top of the deaerator and has an attached gas pressure relief valve attached as well as a pressure gauge 80 for ensuring safe pressure within the deaeration device is maintained below 9 Bar, and preferably between 2 and 6 Bar.

FIG. 9b illustrates the principle of operation of the deaerator. Slurry is pumped from the reactor or soaking tank 25 and into the inlet 60. The slurry rises through the inner tube 55 and fills the deaerator. In recirculation mode, shown on the left, the outlet 65 to the two-stage compression device is closed and the recirculation outlet 70 is opened.

In compressor operation mode, the recycle outlet is partially opened and the compressor outlet is fully opened. Slurry flows up the inner tube and cascades over the sides filing the bottom of the annular space and exiting to the compressor. The headspace above the top of the inner tube accumulates the entrained gases from the slurry and is vented to operate at a back pressure of 2 to 6 Bar, keeping the liquid flowing to the compressor and allowing the entrained gases to be released to the atmosphere.

High Pressure Compression-‘Cell Bursting’

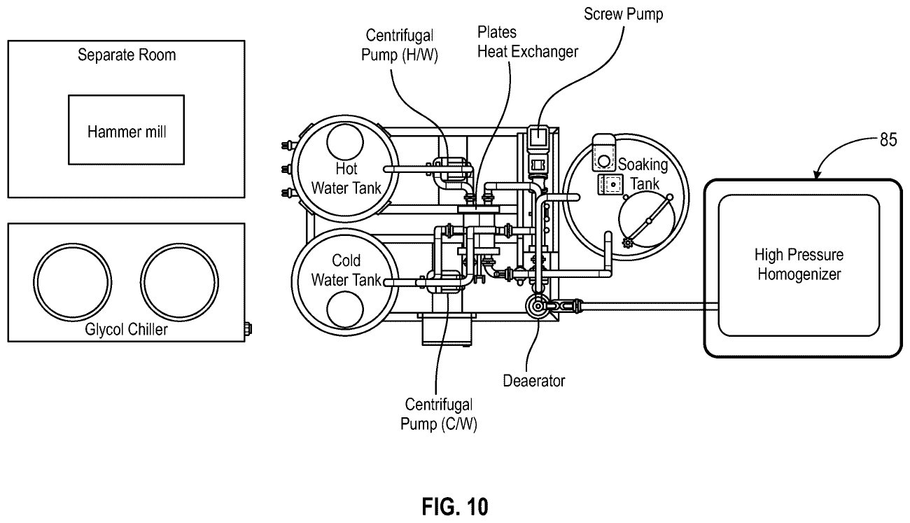

FIG. 10 illustrates where the two-stage high pressure compression device 85 is located in the plant according to the example.

Cell-bursting (or lysis via cavitation) is the ultimate and critical step in the process according to the invention. This occurs in the compression device 85. In this example, the compression device is a modified Ultra High Pressure Homogenizer (HPH). Such HPH's are commonly used to homogenise immiscible liquids into an emulsion, operating like a positive displacement pump with a valve. The internal pistons forces fluids at high pressures through a small orifice. This increases velocity and decreases pressure causing a turbulence/pressure differential that disrupts and disperses particles.

An example of the type of HPH known in the art is the SPX FLOW APV Gaulin & Rannie Homogeniser, as supplied by SPX Flow Technology of Delavan, WI USA, which has been modified for use in this example.

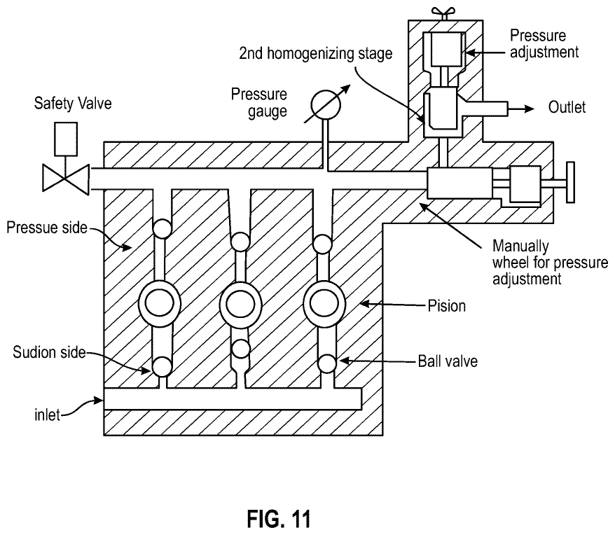

The modified HPH 85, as used in this example of the inventive process, operates on similar principles to a normal HPH, but has a two-stage operation, as illustrated in FIG. 11. In FIG. 11 it can be seen that there are two successive compression chambers, the first operating at higher pressure (typically 400-3000 Bar). The slurry proceeds directly from the high pressure chamber to a second chamber operating at a lower pressure (typically atmospheric to 200 Bar). The sudden pressure drop between the first and second chambers contributes a pressure shock to the cell walls of the material in the slurry and causes them to burst open via cavitation as the water molecules that are forced into the cell at higher pressure rapidly expand at the lower pressure, thereby lysing the cell. This releases functional and nutritional compounds without heat damage (as the operation may be conducted at lower than 70° C. and more typically at or below 60° C.).

At a pressure drop from 900 Bar to a lower pressure, particularly to 0-100 Bar, the cells of most organisms will burst due to this pressure swing cavitation effect. This means that living organisms (such as bacteria and even spores) will also be destroyed (causing a sterilisation effect), and vegetal organisms will release their intra cellular materials, such as but not limited to proteins, cellulose, aromatic alcohol and others.

From here the burst cells, and released materials, are then sent to further processing or packing, depending on the use they are intended for.

The inventors have conducted experiments on various plant and animal cell-based materials using the process according to the invention. The results of these are given in Table 1 below. For each product processed, there is given the ground slurry viscosity according to known observation/publication; the measured viscosity pre-processing; the post processing slurry viscosity; and the dilution factor.

TABLE 1
Viscosity Change Data

| | | Measured | Measured | |
|---|---|---|---|---|
| Product | Standard | Pre-Processing | Post-Processing | Dilution factor |
| Soy Beans | 36 | 36 | 7.0-8.0 | 5 |
| Soy Beans + husks | 40 | 37.4 | 7.6-8.2 | 5 |
| Sorghum | 6.5-12 | 9.1 | 10 | 5 |
| Lupin | 40 | 41 | 8.6 | 5 |
| Oats | 13 | 13.1 | 3.0 | 5 |
| Canola seeds | 17-26 | 20 | 4.7 | 5 |
| Cannabis | 25 | 25 | 5.2 | 5 |
| Dried Kelp chips | 26 | 26 | 5.7 | 4.5 |
| Whole Egg + shells | 12.5 | x | x | x |
| Feathers washed | 85 | 85 | 8.6 | 10 |
| Cocoa powder | 15-20 | x | x | x |
| Coffee beans reworked | 13 | x | x | x |
| Chillies ground | 21.2 | 21.2 | 4.3 | 5 |
| Maize ground + cob | 16 | 15 | 3.1 | 5 |
| Dried Garlic | 4.5 | 4.1 | 1.4 | 3 |
| Dried Turmeric | 6.3 | 6.0 | 1.25 | 5 |
| Whole Pineapple/ | x | x | x | x |
| Oranges/Butternut | | | | |
| Dried Mushroom | 19-39 | 25 | 5.1 | 5 |
| Dried Spirulina | 51-71 | 60 | 12.1 | 5 |
| Algae | 40-60 | x | x | x |
| Mung beans | 21-31 | 26 | 5.3 | 5 |
| Strawberry | 0.7 | 0.7 | 0.26 | 3 |
| Blueberry | 5 | 5 | 1.6 | 3 |
| Buchu herb | x | x | x | x |
| Bay-leaves | x | x | x | x |
| Peanuts | 28.5 | 26 | 6.6 | 4 |
| Apple core | 1.1 | 1.0 | 0.25 | 4 |
| Orange peel | 5.8 | 6 | 1.6 | 1 |
| Watermelon rind | 11.21 | 11 | 2.8 | 4 |
| Broccoli | 3 | 3 | x | x |
| Broccoli leaves/stems | 24-26 | 25 | x | x |
| Black pepper | x | x | x | x |
| Red & Green peppers | 0.99 | x | x | x |
| Carrots | 9 | 10 | x | x |
| Potato | x | x | x | x |
| Potato skins | 3 | x | x | x |
| Sweet potato | 9 | 9 | x | x |
| Sweet potato leaves | 25.5-29.8 | x | x | x |
| Pomegranate | 8 | x | x | x |
| Millet | 12-16 | 15 | 3 | 5 |
| Grape vine shoots | x | x | x | x |
| Rice flour | 11.2 | 12 | 3 | 4 |
| Linseeds | 35 | 35 | 7 | 5 |
| Chia seeds | 19 | x | x | x |
| Chick pea | 17-30 | 22 | 4.4 | 5 |
| Flax seeds | 18 | 16 | 3.3 | 5 |
| Hops | 22-30 | x | x | x |
| Banana skins | 6-9 | x | x | x |
| Vanilla pods | x | x | x | x |
| Invert glucose/fructose | x | x | x | x |
| Balsamic | x | x | x | x |
| Grape pips, ground | 11 | 11 | x | x |
| Red grape skins | x | x | x | x |
| Beetroot | 13 | x | x | x |
| Guava | 13 | x | x | x |
| Avocado, pips ground | 6 | x | x | x |
| Moringa leaves | 23-30 | x | x | x |
| Kiwi fruit | 4 | x | x | x |
| Tomatoes | 18 | x | x | x |
| Leaks | x | x | x | x |
| Flower petals | x | x | x | x |
| Eucalyptus leaves | 13.8 | x | x | x |
| Sugar bagasse | 1-3 | x | x | x |
| Tomato & Tobacco leaf | 20 | x | x | x |

For the above experiments, analysis was carried out on each variety of feed material to establish the viscosity specification and the fineness of grind, prior to cell bursting. Post cell bursting, the microscopic extent of cell rupture and stability under chilling and centrifugation was tested. The moisture variation was determined by the required viscosities that enabled efficient screw pumping and deaeration. This ranged between 22% solids and 66%.

Microscopic examinations of all the samples with regard to residue fibre was undertaken and it was determined that complete cell burst lysis would have been achieved when no microscopic fibres could be seen under 400× magnification.

The following process conditions applied;

Size reduction parameters of material ex-bowl chopper, butcher's mincer-mixer: all <0.8 mm

Ex-hammer mill, colloid mill, sugar mill, roller mill: all <0.8 mm

Soaking tank concentration of material, viscosity of not more than 20,000 centipoise, (dilution of 1:3-1:5). Residence time of between 1 hour and 3 hours.

Target temperature pre-homogenizing: not to be higher than 16° C. Treatment temperatures can vary between 60° and 85° C.

Compression pH was neutral or naturally acidic. Treatment pH may need to be adjusted to between 3.5 to 8 and back to neutral 7.0 if de-bittering is necessary. Protection of the proteins is therefore essential so Potassium Hexa-Meta Phosphate is used to neutralise pH as required.

De-aeration pressure: conducted between 2 Bar and 9 Bar, preferably 6 Bar. Inlet pressure setting for material slurry: 9 Bar pressure.

Eccentric screw pump flow rate: approximately 600 l/hr and up to 1500 l/hr.

Outlet target pressure approximately 600 Bar.

Compression: 1st stage pressure settings at 900-1500 Bar; 2nd stage at 0-350 Bar pressure.

Homogeniser source: Gaulin and Rannie homogeniser from the APV group (customised twin head as per the invention described above).

### Example 1—Oat Concentrate

The process and compression system according to the invention was used to make an oat milk concentrate product.

The composition is given below in table 2.

TABLE 2
Oat Concentrate Composition

| Raw Material | Weight (kg) | Percentage |
|---|---|---|
| Oats | 50 | 19.99 |
| Water | 150 | 59.97 |
| Citric Acid | 0.12 | 0.05 |
| GA 400 Enzyme Alpha | 25 | 10 |
| Amylase | | |
| Alpha Classic Alpha | 25 | 10 |
| Amylase | | |
| TOTAL | 250.12 | 100 |

Step 1 Liquefaction: place 150 L of water in the bioreactor and heat to 80-85° C., adjust pH to 5.5-6.0 by adding citric acid (0.05%). Add alpha amylase enzyme (such as Alpha Classic supplied by Connell) 0.5-1.0 kg/tonne of Oat weight.

Switch on stirrer and add ground oat flour slowly (20-30% solids based on viscosity). Hold in tank for 1-2 hours until starch iodine test is negative.

Step 2 Saccharification: Lower temperature to 60-65° C., then add GA 400 alpha amylase enzyme (as supplied by Connell) at 0.5 kg/tonne of oat weight.

Hold for 1-3 hours in tank depending on desired flavour profile.

Then cool to 5-25° C. for 30 minutes (or until desired temperature is reached) and a pH of about 6.0.

The resulting slurry is then run through the deaeration and HPH stages as per the description above to produce an oat concentrate with cell bursting at 1st/2nd stage pressures of 1200 Bar and 0 Bar respectively.

The product has an Off white, creamy colour with a nutty sweet oat aroma and a nutty oat flavour with a sweet aftertaste. The texture is smooth, with medium consistency.

### Example 2—Oat Milk Product

An oat milk product can then be made from the oat concentrate, as per the ingredients list in table 3 below.

TABLE 3
Oat Milk Composition

| Raw Material | Weight (kg) | Percentage |
|---|---|---|
| Oat concentrate per | 250 | 25 |
| Example 1 | | |
| Salt | 2.0 | 0.20 |
| Coconut Oil | 4.0 | 0.40 |
| Calcium carbonate | 0.2 | 0.02 |
| Di-potassium phosphate | 0.5 | 0.05 |
| TOTAL | 1000 | 100 |

In the blender add oats, water, calcium carbonate, dipotassium phosphate, salt and blend until well emulsified. Add coconut oil to mixture while blending with a high shear blender.

Heat to 95° C. for 5 min, then decant into airtight container and store <4° C.

The properties of the oat milk produced are outstanding, particularly in terms of its frothing ability, indicating that the proteins of the oat grain have been effectively extracted from the cells with a less deleterious effect on their functionality than traditional methods of manufacture. The flavour of the oat milk is very good and it provides a superior alternative to currently available commercial oat milks.

It will be appreciated by those skilled in the art that the above described embodiment is merely one example of how the inventive concept can be implemented. It will be understood that other embodiments may be conceived that, while differing in their detail, nevertheless fall within the same inventive concept and represent the same invention.

1. A process for effecting lysis of cells of plants material or parts thereof; the process comprising:
obtaining the plant material at a target particle size;
combining the plant material in a fluid medium in a vessel;
subjecting the plant material to a set of predetermined processing conditions;
deaerating the plant material to substantially reduce or remove entrained gases; and
introducing the plant material into a two-stage compression device, within which it is subjected to a first pressure in a high fluid pressure compression first stage, and subsequently to a second pressure in a rapid decompression second stage, wherein the first pressure is substantially higher than the second pressure thereby subjecting the plant material to cavitation of water inside the cell wall causing cell wall rupture.

2. The process of claim 1, wherein the target particle size of the plant material ranges between 400 and 2000 microns.

3. The process of claim 1, wherein the set of predetermined processing conditions is dependent on raw materials used and includes one or more of: temperature, temperature profile, pH, viscosity, and time.

4. The process of claim 3, wherein the set of predetermined processing conditions includes a temperature below 60 C.

5. The process of claim 1, wherein the deaeration action and the compression process occur concurrently via a partial recycling of compressed material into the vessel from the outlet of the deaeration device.

6. The process of claim 1, wherein the first pressure is between 400 Bar and 3000 Bar.

7. The process of claim 6, wherein the first pressure is between 900 Bar and 2000 Bar.

8. The process of claim 1, wherein the second pressure is between atmospheric pressure and 400 Bar.

9. The process of claim 7, wherein the ratio of the first pressure to the second pressure is 4:1 or greater.

10. The process of claim 1, wherein the first pressure is approximately 900 Bar and the second pressure is approximately 200 Bar.

11. The process of claim 1, wherein the first pressure is in the range of approximately 600-1500 Bar and the second pressure is in the range of approximately 0-350 Bar.

12. The process of claim 1, wherein the deaeration device comprises a substantially vertical pressure vessel having an outer tube and an open inner annular tube, wherein the inner tube extends between 65% and 75% of the length of the outer tube from an inlet end of the deaeration device; and wherein the plant material entering the deaeration device is directed internally of the inner tube.

13. A two-stage high fluid pressure compression device for effecting lysis of cells of plant material, the device comprising:
a first stage for subjecting the plant material to a first pressure; and
a second stage for subjecting the plant material to a second pressure that is substantially lower than the first pressure,
wherein the pressure differential between the first and second stages causes cavitation of water inside the cell wall of the plant material, leading to cell wall rupture.

14. The device of claim 13, wherein the first pressure is between 400 Bar and 3000 Bar.

15. The device of claim 14, wherein the first pressure is between 900 Bar and 2000 Bar.

16. The device of claim 13, wherein the second pressure is between atmospheric pressure and 400 Bar.

17. The device of claim 15, wherein the ratio of the first pressure to the second pressure is 4:1 or greater.

18. The device of claim 13, wherein the first pressure is approximately 900 Bar and the second pressure is approximately 200 Bar.

19. The device of claim 13, wherein the first pressure is in the range of approximately 600-1500 Bar and the second pressure is in the range of approximately 0-350 Bar.

20. The device of claim 13, wherein the two-stage high fluid pressure compression device is coupled at its inlet to a deaeration device that comprises a substantially vertical pressure vessel having an outer tube and an open inner annular tube, wherein the inner tube extends between 65% and 75% of the length of the outer tube from an inlet end of the device; and wherein the plant material entering the device is directed internally of the inner tube.

21. A food product comprising plant material processed by a method comprising:
obtaining the plant material at a target particle size;
combining the plant material in a fluid medium in a vessel;
subjecting the plant material to a set of predetermined processing conditions;
deaerating the plant material to substantially reduce or remove entrained gases; and
introducing the plant material into a two-stage compression device, within which it is subjected to a first pressure in a high fluid pressure compression first stage, and subsequently to a second pressure in a rapid decompression second stage, wherein the first pressure is substantially higher than the second pressure thereby subjecting the plant material to cavitation of water inside the cell wall causing cell wall rupture.

22. The food product of claim 21, wherein the target particle size of the plant material ranges between 400 and 2000 microns.

23. The food product of claim 21, wherein the first pressure is between 400 Bar and 2000 Bar, and the second pressure is between atmospheric and 400 Bar.
